(19) United States
(12) Patent Application Publication
(10) Pub. No.: US 2003/0093045 A1
(43) Pub. Date: May 15, 2003

(54) Absorbing article having zoned areas of hydrophilicity

(76) Inventor: Carol L. Erdman, Duluth, GA (US)

Correspondence Address: Christopher C. Campbell, Esq., Hunton & Williams, 1900 K Street, NW, Suite 1200, Washington, DC, 20006-1109, US

(21) Appl. No.: 09/987,196

(22) Filed: Nov. 13, 2001

(51) Int. Cl. A61F 13/15

(52) U.S. Cl. 604/367; 604/385.08

(57) ABSTRACT

An absorbent article that provides leakage protection and/or skin protection, as well as methods for using and preparing same and novel compositions and composites for use in same, is disclosed. The absorbent article comprises a selectively-permeable topsheet; a substantially impermeable backsheet; an absorbent core positioned between said selectively-permeable topsheet and said substantially impermeable backsheet. The selectively-permeable topsheet has a treated hydrophilic zone and an untreated hydrophobic zone.

### FIELD OF THE INVENTION

This invention relates to the field of absorbent articles in general, and more particularly to absorbent articles that provide leakage protection and/or skin protection, as well as methods for using and preparing same, and novel compositions and composites for use in said absorbent articles.

### BACKGROUND OF THE INVENTION

Disposable absorbent articles typically include a moisture-impervious backing sheet, an absorbent pad, and a moisture permeable liner sheet (e.g., topsheet) that contacts the body of a person wearing the article. In addition, elasticized regions are provided around the edges of the article to secure the article about the waist and legs of a wearer. Diapers typically further comprise opposed front and rear waist portions defining a waist opening, a crotch portion disposed there between, and a pair of elastically contractible leg openings along the side edges of the crotch portion. Disposable diapers having elasticized margins for placement about the legs of a wearer are disclosed in U.S. Pat. Nos. 4,050,462 and 5,092,861. An absorbent article having elasticized side margins and waist band margins are shown in U.S. Pat. No. 4,300,562.

Despite previous advancements in the field of absorbent articles, persons of ordinary skill in the art continue their efforts to produce more comfortable garments which are better able to contain urinary and fecal excretions. For instance, problems with prior diaper designs include leakage of urinary or fecal material from the garment. Prolonged contact of liquid or semi-solid excreta with the skin of the wearer is also a continuing problem in the art. For example, the moisture vapor and heat generated by the bodily exuded trapped within a diaper may lead conditions adjacent to wearer's skin which promotes skin irritation, infection, and the like. Although the topsheet, as described above, is generally effective in allowing the passage of bodily exude outwardly, the moisture permeable nature of topsheets results in leakage of liquids. Various approaches have been attempted to address the lateral leakage of liquids from absorbent articles.

For example, U.S. Pat. No. 6,114,596 to Nayak et al. discloses a breathable diaper, feminine hygiene, or like disposable sanitary product having a cloth-like outer surface and including a plurality of materials from the skin-facing side outwardly, a selectively-permeable topsheet, a core, an optional barrier, and a backsheet. The disclosed topsheet is formed of liquid- and vapor-permeable hydrophilic material and the core is formed of highly absorbent material disposed outwardly of the selectively-permeable topsheet for absorbing liquid received through the selectively-permeable topsheet. The disclosed core has an inter surface in liquid communication with the selectively-permeable topsheet and an outer surface. The disclosed optional barrier is formed for a multi-layer non-woven material which is hydrophobic and vapor-permeable for limiting the outward escape of liquid therethrough while enabling the outward escape of heat and water vapor therethrough. The disclosed barrier has a base disposed adjacent the core outer surface. The disclosed backsheet is formed of a multi-layer non-woven material which is hydrophobic and vapor permeable for limiting the outward escape of liquid therethrough while enabling the outward escape of heat and water vapor therethrough. This backsheet is disclosed as being disposed at least partially as an outer surface of the diaper.

U.S. Pat. No. 6,017,336 to Sauer discloses an absorbent article which includes a pair of compression resistant containment barriers which are configured to inhibit the lateral flow of fecal exudates along the surface of the absorbent article. The containment barriers are disclosed as being laterally spaced apart to provide a void space between the wearer's back side and the surface of the absorbent article for containing body exudates. Each containment barrier is disclosed as defining a width to height ratio of at least about 0.5 and a compression resistance of at least about 50%. The absorbent article is disclosed as optionally including a containment dam which is located on the body facing surface of the absorbent article and which is configured to inhibit a longitudinal flow of fecal exudates along the surface of the absorbent article.

U.S. Pat. No. 5,597,906 to Roe, et al. discloses an absorbent article comprising a liquid pervious topsheet, a liquid pervious backsheet joined to at least a portion of the topsheet, an absorbent cord disposed between at least a portion of the topsheet and the backsheet, and a waste management element disposed in at least a portion of the crotch region. The waste management element preferably has an acceptance under pressure value of greater than about 0.50 grams of a viscous fluid bodily waste per square inch of the waste management element millijoule of energy input. The waste management element is also disclosed as having a storage under pressure values of at least about 0.70 grams of the viscous fluid bodily waste per square inch of the waste management element. The waste management element is also disclosed as optional having an Immobilization Under Compressed Inversion valve of greater than about 70% of the viscous fluid bodily waste accepted by the waste management element. The waste management is disclosed as being located anywhere in the article, including the crotch region.

U.S. Pat. No. 5,941,864 to Roe discloses a disposable absorbent article, such as a diaper having a first topsheet with apertures large enough for low-viscosity fecal material to pass through to a fecal material storage element. The fecal material storage element is disclosed as immobilizing the fecal material in position for dewatering, so that the liquid components of the fecal material are absorbed by the core and solid components of the fecal material are separated from the liquid components, to provide for easier cleaning of the wearer when the soiled disposable absorbent article is removed.

U.S. Pat. No. 5,941,862 to Haynes et al. discloses absorbent structures comprising a first layer, a second layer juxtaposed in facing relation with said first layer, wherein at least one of the layers is fluid pervious. These structures are disclosed as having a continuous region between said first and second layers comprising hydrogel-forming polymer that is substantially uniformly distributed throughout the region. The continuous region is disclosed as at least partially surrounding multiple, spaced apart zones between said layers, which zones are substantially devoid of hydrogel-forming absorbent polymer. The first and second layers are disclosed as being bonded together such that said hydrogel-forming absorbent polymer is substantially immobilized when in dry state, an preferably at sites within plurality of the zones.

U.S. Pat. No. 5,558,655 to Jezzi et al. discloses in absorbent article, such as diaper, which comprises a composite structure of a “two-dimensional” or very flat, apertured film or nonwoven layer, in combination with a fluid transferring layer and a superabsorbent polymer-containing laminate, and a absorbent core below the laminate to achieve superior dryness. The superabsorbent polymer-containing laminate is disclosed as containing airlaid fibrous components and superabsorbent polymers and as swelling to at least three times its dry caliper upon fluid introduction in order to achieve movement of the coverstock away from the absorbent core which permits the coverstock to remain relatively dry and avoids rewetting.

U.S. Pat. No. 6,040,251 to Caldwell discloses barrier webs at a certain desirable physical qualities such as water resistance, increased durability, improved barrier qualities and the like. Barrier webs are disclosed as comprising a web that has been treated with a curable shear thinned thixotropic polymer composition which is adapted to be substantially impermeable to liquids, permeable to gases and impermeable to microorganisms. Further, the barrier webs are disclosed as being either impermeable to all microorganisms or impermeable to microorganisms of certain sizes. Also disclosed are fabrics that are capable of either selectively binding certain microorganisms, particles, or molecules depending upon what binding partners are incorporated into the polymer before application to the fabric.

U.S. Pat. No. 5,945,175 to Yahiaoui et al discloses a coated porous substrate composed of a hydrophobic polymer which is substantially uniformly coated with a hydrophilic polymeric material. The substrate may be a sheet-like material, examples of which are forms, fibers, and fibrous webs. The fibrous webs are disclosed as desirably being nonwoven webs. The coating on the substrate is disclosed as being durable to an aqueous medium at a temperature in a range of from about 10° C. to about 50° C. and does not significantly suppress the surface tension of an aqueous medium with which the coated substrate may come in contract. The hydrophobic polymer is disclosed as being a polyolefin, such as polyethylene or polypropylene. The hydrophilic polymeric material is disclosed as being a polysaccharide or a modified polysaccharide.

U.S. Pat. No. 6,117,121 to Faulks et al. discloses an absorbent article including an absorbent core located between a bodyside liner and an outer cover. The absorbent article is disclosed as having a leg cuff mounted to a base structure in the crotch portion thereof. The leg cuffs are disclosed as being partially stretched when attached to respective longitudinal side portions near the crotch portion of the absorbent article.

U.S. Pat. No. 6,107,539 to Palumbo et al. discloses disposable absorbent articles comprising a backsheet, a topsheet, a fluid acquisition/distribution region and at least one fluid storage region, said article having a total product acquisition performance of more than 3.75 ml/sec in the first gush and more than 0.5 mil/sec in the fourth gush and an in bag stack height of less than 9.9 mm, characterized in that said topsheet allows it to retain no more then 0.25 g of fluid as measured by the topsheet-on-acquisition-material-wetness test, and that said acquisition/distribution region has a drip capacity of at least 5.0 grams of fluid per gram of material.

U.S. Pat. No. 5,935,118 to Gryskiewicz et al. discloses an absorbent article that includes a garment shell and at least one liquid containment beam formed of an absorbent material. The liquid containment beam has an attachment edge bonded to the garment shell so that the containment beam can lie against the garment shell and also pivot about an axis defined by the attachment edge. For comfort during use, the containment beam desirably has a width to thickness ratio of at least about 3:1. In particular embodiments, the absorbent article includes pairs of inner and outer containment beams, with the inner containment beams adapted to lie against the garment shell and the outer containment beams adapted to lie against the inner containment beams.

U.S. Pat. No. 6,087,550 to Anderson-Fischer et al. relates to water responsive thermoplastic compositions and articles constructed thereof. This invention particularly relates to thermoplastic copolyester compositions useful for the manufacture of disposable articles such as disposable diapers and feminine napkins. More particularly, this invention relates to thermoplastic copolyester compositions that are useful as a raw material in the manufacture of nonwovens, barrier films or coatings, as well as for various improved hot melt adhesive compositions useful for incorporating hydrophilic features into disposable articles.

U.S. Pat. No. 6,120,783 to Roe et al. discloses web materials which have two or more skin care compositions disposed thereon. The skin care compositions are transferable to the wearer's skin by normal contact and/or wearer motion and/or body heat. The skin care compositions disclosed in the present invention are selected to maintain and/or improve the skin health of the wearer upon transfer during use, for example, to provide a skin protective barrier or a therapeutic benefit; to minimize the abrasion between the cuffs and skin in the area where the cuffs contact the wearer's skin, resulting in less skin irritation; to improve BM clean up on the skin, or to improve the barrier properties of the cuffs. Web materials of the present invention have a wide range of potential uses in both durable and disposable articles, but are particularly well suited for use in disposable absorbent articles such as disposable diapers, incontinent briefs, training pants, sanitary napkins, and the like.

U.S. Pat. No. 5,683,809 to Freeman et aL discloses protective articles such as diapers, having filmless hydrophobic barrier elements such as cuffs and backing sheets. The barrier cuffs—which can be, for instance, leg cuffs and waistbands—and the backing sheets can be provided from fabrics having a fabric weight of at least 10 gsy.

U.S. Pat. No. 5,928,209 to Bodford et al. discloses a breathable diaper, feminine hygiene, or like disposable sanitary product construction includes a plurality of materials including, from the skin-facing side outwardly, a topsheet of liquid- and vapor-permeable hydrophilic material. A core of highly absorbent material is disposed outwardly of the topsheet for absorbing fluid received through the topsheet, the core having an inner surface in fluid communication with the topsheet. A backsheet is disposed at least partially as an outer surface of the construction and is formed of a pouch defined by two layers of a non-woven hydrophobic and vapor-permeable material, with absorbent or superabsorbent particles therebetween, so that the backsheet limits the outward escape of liquid therethrough while enabling the outward escape of heat and water vapor therethrough. Each backsheet layer is an at least two-layer spunbound-meltblown SM or an at least three-layer spunbond-meltblown-spunbond SMS.

U.S. Pat. No. 5,990,377 to Chen et al. discloses a dual-zoned, three-dimensional, resilient absorbent web is disclosed which is suitable as body-side liner for absorbent articles such as feminine pads, diapers and the like. When used as a liner in absorbent articles, the dual-zoned web combines the advantages of apertured films and soft, nonwoven cover layers in one structure while still being inherently hydrophilic. The liner comprises a web of wet-resilient, hydrophilic basesheet having a three-dimensional topography comprising elevated regions onto which hydrophobic matter is deposited or printed and a plurality of spaced apart depressed regions. In a preferred embodiment, the hydrophobic matter applied to the elevated regions of the basesheet comprises hydrophobic fibers in a contiguous nonwoven web which has been apertured or provided with slits or other openings, such that the apertures or openings overlay a portion of the depressed regions. The elevated hydrophobic regions enhance dry feel and promote fluid flow toward the lower hydrophilic regions, which comprise the exposed depressed regions of the basesheet. The basesheet is preferably in liquid communication with underlying absorbent material, most preferably a stabilized airlaid cellulosic material or compressed stabilized fluff such that the absorbent material can wick fluid out of the basesheet by capillary action. When soft, hydrophobic fibers are deposited on the elevated regions, the liner also has a soft, cloth-like feel in addition to a dry feel in use.

U.S. Pat. No. 6,120,488 to VanRijswijck et al. discloses an absorbent article, such as a diaper, containing cuffs and a topsheet with one or more skin care compositions disposed thereon. The skin care compositions are transferable to the wearer's skin by normal contact and/or wearer motion and/or body heat. The skin care compositions disclosed in the present invention are selected to maintain and/or improve the skin health of the wearer upon transfer during use, for example, to provide a skin protective barrier or a therapeutic benefit; to minimize the abrasion between the cuffs and skin in the area where the cuffs contact the wearer's skin, resulting in less skin irritation; to improve BM clean up on the skin, or to improve the barrier properties of the cuffs.

U.S. Pat. No. 5,817,079 to Bergquist et al. discloses in absorbent products, such as sanitary napkins, discreet areas of dry fibrous materials such as fluid-repellent materials are precisely placed in various planes within the product so as to provide barriers to bodily fluid leakage from the product. In a preferred embodiment, hydrophobic fibers are placed around the periphery of a central absorbent area of an absorbent product to discourage and/or prevent side or end leakage from the product.

U.S. Pat. No. 5,830,201 to George et aL discloses a flushable diaper for use on the body of an infant or adult. An envelope has inner and outer walls which are secured together at their outer periphery to form an inner cavity. The outer wall is comprised of a hydrophobic outer layer together with a hydrophilic inner layer. The inner wall is comprised of a hydrophobic outer layer together with a hydrophilic inner layer. The inner and outer walls are separated by an interior cavity which contains one or more containers. The container is comprised of either a single layer or a laminated wall which forms a chamber that encloses a charge of water. The laminated wall is comprised a hydrophobic inner laminate and a hydrophilic outer laminate. When manually ruptured, the containers release water into the double walled envelope, which causes disintegration of the hydrophilic inner layers of the double walls, along with the hydrophobic outer layers. An absorbent member is carried on the inner wall of the envelope, and the absorbent member comprises a fluid pervious cover which encloses an inner body that is comprised of a material which is sufficiently hydrophilic to absorb urine and other fluids from the body while maintaining structural integrity of the inner body and to also absorb water in an effective amount to cause the inner body to disintegrate into small pieces.

U.S. Pat. No. 5,885,266 to Chihani et al. discloses an absorbent article such as a diaper, an incontinence guard or a sanitary napkin includes a liquid-perrneable outer sheet, a liquid-impermeable bottom sheet and an absorbent body placed therebetween. The outer sheet and the absorbent body are at least partially joined together with a hydrophilic glue.

As is apparent from the foregoing, each of the prior references present a variety of means for controlling leakage in absorbent garments. However, all of these proposed means are deficient in terms of effectiveness and low product quality, mechanical complexity in design, and/or associated cost inefficiencies.

In view of the deficiencies of the various products and processes disclosed in the above discussed references, it is highly desirable to provide cost-efficient absorbent articles that display superior leak protection, as well as novel compositions and composites for use in said absorbent articles. Further, it is highly desirable to provide a cost-efficient process for producing absorbent articles having superior leak protection. Moreover, it is also highly desirable to provide cost-efficient absorbent articles, and methods for preparing such articles, that confer secondary benefits of skin wellness to the users of said articles, in addition to superior leak protection.

### SUMMARY OF THE INVENTION

The present invention provides cost-efficient absorbent articles, and novel compositions and composites for use in same, that display unexpectedly superior leak protection. Further, the present invention provides a cost-efficient process for producing absorbent articles having superior leak protection. Moreover, the present invention provides cost-efficient absorbent articles, methods for preparing and using such articles, and novel compositions and composites for use in same, that confer secondary benefits of skin wellness to the users of said articles in addition to superior leak protection. Various embodiments of the present invention are described below.

In one embodiment, the present invention provides an absorbent article comprising: a selectively-permeable topsheet; a substantially impermeable backsheet; and an absorbent core disposed between said selectively-permeable topsheet and said substantially impermeable backsheet; wherein the selectively-permeable topsheet comprises at least one treated hydrophilic zone and at least one non-treated hydrophobic zone.

In a further embodiment, the present invention provides an absorbent article comprising: a selectively-permeable topsheet; a substantially impermeable backsheet; and an absorbent core disposed between said selectively-permeable topsheet and said substantially impermeable backsheet; wherein the selectively-permeable topsheet comprises at least one treated hydrophilic zone and at least one non-treated hydrophobic zone, said treated hydrophilic zone comprising a surfactant and a skin wellness ingredient; and wherein the treated hydrophilic zone is defined by an area that corresponds to a predetermined insult point or plurality of insult points.

In a still further embodiment, the present invention provides an absorbent garment comprising: a front and a rear waist portion cooperating to form a waist opening; a crotch region formed between said front waist portion and said rear waist portion; a pair of leg openings on opposed sides of the crotch region; and a selectively permeable topsheet, a substantially impermeable backsheet and an absorbent core positioned between said selectively-permeable topsheet and said backsheet; wherein the selectively-permeable topsheet comprises at least one treated hydrophilic zone corresponding to a portion or plurality of portions at the crotch region and at least one non-treated hydrophobic zone.

In an even further embodiment, the present invention provides a method for preparing an absorbent article comprising: disposing a composition on a nonwoven at one or more predetermined area corresponding to an insult point or plurality of insult points to form a selectively-permeable topsheet having a treated hydrophilic zone or plurality of treated hydrophilic zones corresponding to the one or more predetermined areas, said nonwoven being inherently hydrophobic and comprising fibers or film.

In another embodiment, the present invention provides a composition in an absorbent article for providing leak protection and improved skin wellness comprising: an amount of surfactant sufficient for rendering substantially hydrophilic an inherently hydrophobic material comprising nonwoven fibers or films; and a skin wellness substance selected from the group consisting of Aloe (aloe barbadensis), Bisabolol, Calendula Officinalis, Canadian Balsam (Abies balsamea), Cocoa Butter (Theobroma Cacao seed butter), Eucalyptus leaf oil (Eucalyptus globulus), German chamomile (Matricaria recutica), Gorgonian extract (Pseudotergoria elisabethae), Jojoba, Lanolin, Lavandin (Lavandula X intermedia), Lemon balm (Melissa officinalis), Rosemary (Rosmarinus officinalis), Spike lavender (Lavandula latifolia), Tea tree (Melaleuca alternifolia), True Lavender (Lavandula angustifolia), Vitamin E, Vitamin E-acetate, Witch Hazel (Hamamelis Virginiana) and combinations thereof.

### BRIEF DESCRIPTION OF THE DRAWINGS

FIG. 1 is a topsheet perspective of an absorbent article showing a selectively-permeable topsheet having a treated hydrophilic zone corresponding to a female insult point in accordance with an implementation of the present invention.

FIG. 2 is a topsheet perspective of an absorbent article showing a selectively-permeable topsheet having a treated hydrophilic zone corresponding to a male insult point in accordance with an implementation of the present invention.

FIG. 3 is a topsheet perspective of an absorbent article showing a selectively-permeable topsheet having two treated hydrophilic zones, each corresponding to a female and a male insult point, in accordance with an implementation of the present invention.

FIG. 4. is a topsheet perspective of an absorbent article showing a selectively-permeable topsheet having a treated hydrophilic zone corresponding to a female and male insult point in accordance with an implementation of the present invention.

FIG. 5 is a topsheet perspective of an absorbent article showing a selectively-permeable topsheet having a treated hydrophilic zone corresponding to female and male insult points in accordance with implementations of the present invention.

FIG. 6 is a graph illustrating insult points on diaper brands in a gender-based statistical study.

FIG. 7 is a graph illustrating variation in insult point over time in a gender-based study.

### DETAILED DESCRIPTION OF THE INVENTION

The present invention provides an absorbent article, as well as a method of preparing said absorbent article, a method of using said absorbent article and a composition for use in absorbent articles, having unexpectedly superior properties of leakage protection and/or skin wellness. The present invention can be understood by the disclosure herein and/or by reference to the drawings.

The present invention provides absorbent articles comprising a selectively-permeable topsheet. The term “selectively-permeable topsheet”, as used herein, refers to a topsheet comprising an inherently hydrophobic material which is partially treated at a predetermined zone (including one or more than one contiguous area) with a material that renders the treated portions substantially hydrophilic while the untreated portions remains hydrophobic.

As used herein, the term “absorbent article” refers to articles that absorb and contain exudates, and more specifically refers to articles which are placed against or in proximity to the body of a wearer of the absorbent article to absorb and contain various exudates discharged from the body. A non-exhaustive list of examples of absorbent articles includes diapers, diaper cores, diaper covers, disposable diapers, training pants, feminine hygiene products and adult incontinence products. The term “disposable article” refers to absorbent articles that are intended to be discarded or partially discarded after a single use, i.e., they are not intended to be laundered or otherwise restored or reused. The term “unitary disposable absorbent article” refers to a disposable absorbent article that is essentially a single structure (i.e., it does not require separate manipulative parts such as a diaper cover and insert). As used herein, the term “diaper” refers to an absorbent article generally worn by infants and incontinent persons about the lower torso.

The claims are intended to cover all of the forgoing classes of absorbent articles, without limitation, whether disposable, unitary or otherwise. These classifications are used interchangeably throughout the specification, but are not intended to limit the claimed invention. The invention will be understood to encompass, without limitation, all classes of absorbent articles, including those described above. Preferably, the absorbent core is thin in order to improve the comfort and appearance of a garment. The employance of thin, comfortable garments is disclosed, for example without limitation in U.S. Pat. No. 5,098,423 to Pineiak et al. which is incorporated in its entirety herein by reference.

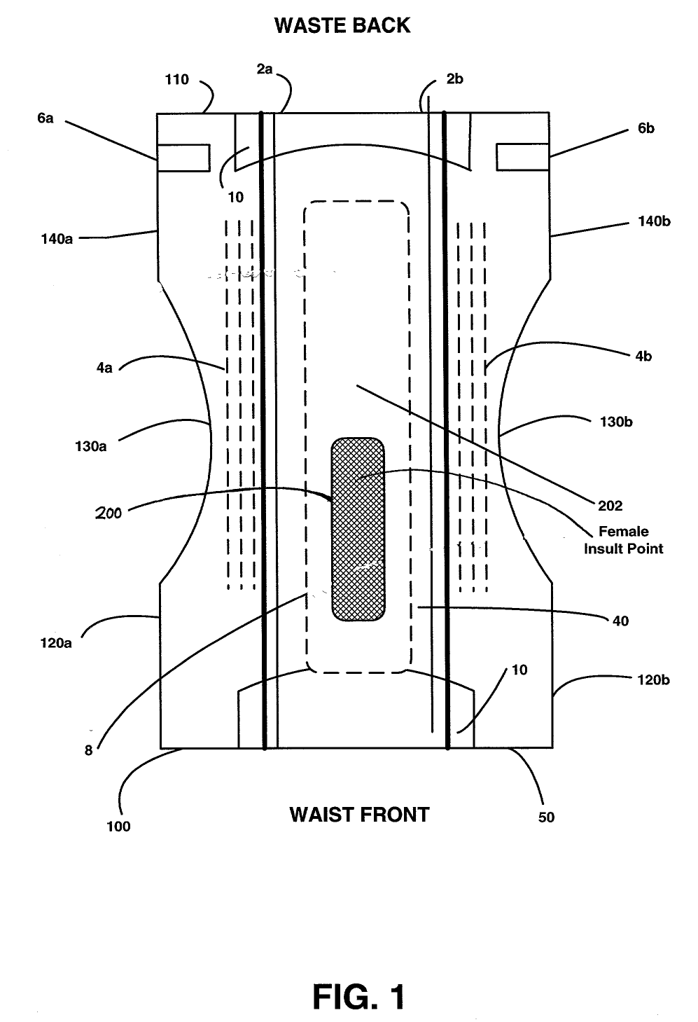

Referring to FIG. 1, an absorbent article (e.g., a diaper) is shown from a topsheet perspective. The absorbent article comprises a front edge 100, a back edge 110, two front side edges 120a and 120b, two back side edges 140a and 140b, and two leg cutout edges 130a and 130b, positioned such that leg cutout edge 130a is disposed between front side edge 120a and back side edge 140a, and leg cutout edge 130b is disposed between front side edge 120b and 140b.

Referring still to FIG. 1, the absorbent article further comprises two inner leg gathers 2a and 2b, each of which extends longitudinally along the topsheet of the absorbent article from the back edge 110 to the front edge 100. The inner leg gathers 2a and 2b are approximately parallel to one another and are each positioned approximately parallel to the front and back side edges 120a, 120b, 140a and 140b in the longitudinal direction of the absorbent article. Further, each inner leg gather is located adjacent to and inward of the inner most portion of the leg cutout edges 130a and 130b.

Referring still to FIG. 1, the absorbent article further comprises two leg elastics 4a and 4b, each of which is positioned approximately adjacent and parallel to each of the inner leg gathers 2a and 2b. Each of the leg elastics is located outward of said inner leg gathers 2a and 2b. Two fasteners 6a and 6b are each located at or near the back side edge of the absorbent article. An absorbent core 8 is shown as being disposed between the topsheet 40 and a substantially impermeable backsheet 50. The absorbent core 8 is positioned longitudinally along the absorbent article. Waist foam 10 is optionally present in the absorbent article and positioned near and adjacent to the front edge 100 and/or back edge 110, disposed between topsheet 40 and the backsheet.

As shown in FIG. 1, a hydrophilic zone 200 is shown being located on the topsheet 40 inward of the front edge 100 and positioned centrally between the two inner leg gathers 2a and 2b, as well as the outside edges of the absorbent core, and corresponding to a female insult point. Also shown adjacent to the hydrophilic zone 200 on the topsheet 40 is a hydrophobic zone 202. The hydrophobic zone 202 encompasses every portion of the topsheet not indicated as the hydrophilic zone 200. The hydrophilic zone 200 is defined as an approximately oval area, for purposes of example only. The hydrophilic zone may be any shape that efficiently provides superior leak protection and absorbency, as could be readily determined by a person of skill in the art. The illustrations provided herein of the hydrophilic and hydrophobic zones is merely illustrative and the present invention contemplates any predetermined placement of the hydrophilic zone in the manner as described herein and the like.

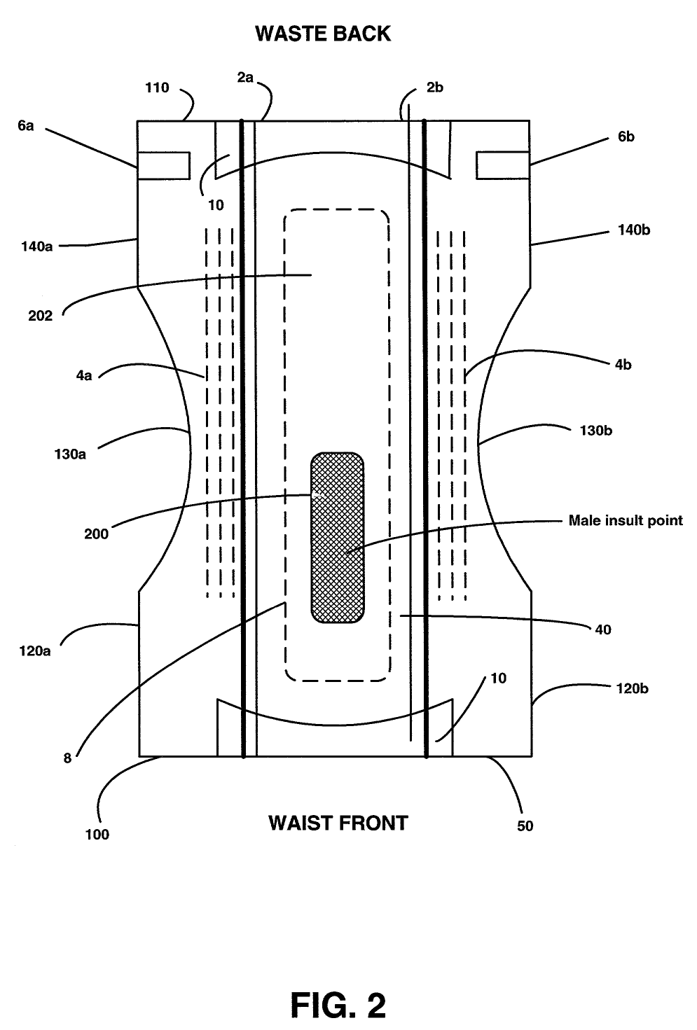

Referring to FIG. 2, an absorbent article (e.g., a diaper) is shown from a topsheet perspective. The absorbent article comprises a front edge 100, a back edge 110, two front side edges 120a and 120b, two back side edges 140a and 140b, and two leg cutout edges 130a and 130b, positioned such that leg cutout edge 130a is disposed between front side edge 120a and back side edge 140a, and leg cutout edge 130b is disposed between front side edge 120b and 140b.

Referring still to FIG. 2, the absorbent article further comprises two inner leg gathers 2a and 2b, each of which extends longitudinally along the topsheet of the absorbent article from the back edge 110 to the front edge 100. The inner leg gathers 2a and 2b are approximately parallel to one another and are each positioned approximately parallel to the front and back side edges 120a, 120b, 140a and 140b in the longitudinal direction of the absorbent article. Further, each inner leg gather is located adjacent to and inward of the inner most portion of the leg cutout edges 130a and 130b.

Referring still to FIG. 2, the absorbent article further comprises two leg elastics 4a and 4b, each of which is positioned approximately adjacent and parallel to each of the inner leg gathers 2a and 2b. Each of the leg elastics is located outward of said inner leg gathers 2a and 2b. Two fasteners 6a and 6b are each located at or near the back side edge of the absorbent article. An absorbent core 8 is shown as being disposed between the topsheet 40 and a substantially impermeable backsheet 50. The absorbent core 8 is positioned longitudinally along the absorbent article. Waist foam 10 is optionally present in the absorbent article and positioned near and adjacent to the front edge 100 and/or back edge 110, disposed between the topsheet 40 and the backsheet 50.

As shown in FIG. 2, a hydrophilic zone 200 is shown being located on the topsheet 40 inward of the front edge 100 and positioned centrally between the two inner leg gathers 2a and 2b, as well as the outside edges of the absorbent core, and corresponding to a male insult point. Also shown adjacent to the hydrophilic zone 200 on the topsheet 40 is a hydrophobic zone 202. The hydrophobic zone 202 encompasses every portion of the topsheet not indicated as the hydrophilic zone 200.

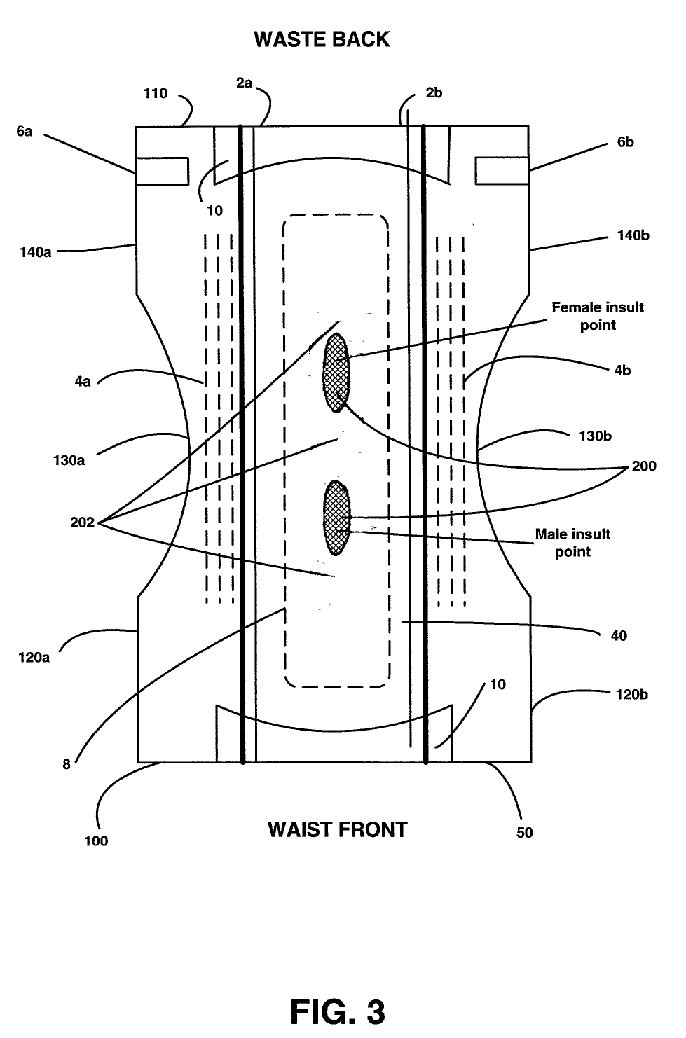

Referring to FIG. 3, an absorbent article (e.g., a diaper) is shown from a topsheet perspective. The absorbent article comprises a front edge 100, a back edge 110, two front side edges 120a and 120b, two back side edges 140a and 140b, and two leg cutout edges 130a and 130b, positioned such that leg cutout edge 130a is disposed between front side edge 120a and back side edge 140a, and leg cutout edge 130b is disposed between front side edge 120b and 140b.

Referring still to FIG. 3, the absorbent article further comprises two inner leg gathers 2a and 2b, each of which extends longitudinally along the topsheet of the absorbent article from the back edge 110 to the front edge 100. The inner leg gathers 2a and 2b are approximately parallel to one another and are each positioned approximately parallel to the front and back side edges 120a, 120b, 140a and 140b in the longitudinal direction of the absorbent article. Further, each inner leg gather is located adjacent to and inward of the inner most portion of the leg cutout edges 130a and 130b.

Referring still to FIG. 3, the absorbent article further comprises two leg elastics 4a and 4b, each of which is positioned approximately adjacent and parallel to each of the inner leg gathers 2a and 2b. Each of the leg elastics is located outward of said inner leg gathers 2a and 2b. Two fasteners 6a and 6b are each located at or near the back side edge of the absorbent article. An absorbent core 8 is shown as being disposed between the topsheet 40 and a substantially impermeable backsheet 50. The absorbent core 8 is positioned longitudinally along the absorbent article. Waist foam 10 is optionally present in the absorbent article and positioned near and adjacent to the front edge 100 and/or back edge 110, disposed between the topsheet 40 and the backsheet 50.

As shown in FIG. 3, a hydrophilic zone 200 is shown as two separate contiguous areas, each located on the topsheet 40 inward of the front edge 100 and positioned centrally between the two inner leg gathers 2a and 2b, as well as the outside edges of the absorbent core, and corresponding to male and female insult points. Also shown adjacent to the hydrophilic zone 200 on the topsheet 40 is a hydrophobic zone 202. The hydrophobic zone 202 encompasses every portion of the topsheet not indicated as the hydrophilic zone 200. The hydrophilic zone can comprise one or more separate contiguous areas, as desired. The placement of such hydrophilic zones is well within the skill of the art, based upon the guidance provided herein.

The insult point may be determined in a variety of ways as would be known to a person of skill in the art. According to a preferred implementation of the present invention, a method of determining insult zones is as follows: Children are diapered and allowed to wear the applied diaper for a pre-determined amount of time to allow the diaper to follow the natural contours of the body and to allow for any sagging or stretching of the diaper that normally occurs. The fasteners of the diaper are gently opened, and with one hand on the outside of the diaper and against the child's buttocks to hold the diaper in place, the front of the diaper is gently peeled back from the child's abdomen to the opening of the urethra. Using a marker pen, a small dot is placed on the inside of the diaper at the point where the urethra would contact the diaper topsheet. The diaper is then completely removed from the child and measurements taken on the diaper to determine the placement of the pen mark.

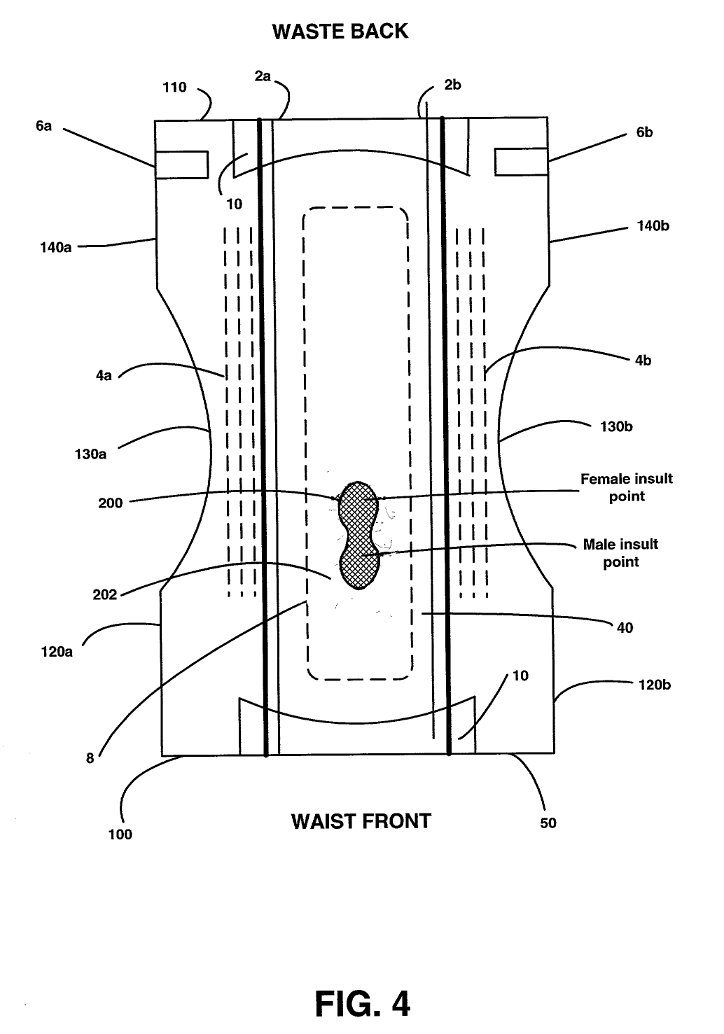

Referring to FIG. 4, an absorbent article (e.g., a diaper) is shown from a topsheet perspective. The absorbent article comprises a front edge 100, a back edge 110, two front side edges 120a and 120b, two back side edges 140a and 140b, and two leg cutout edges 130a and 130b, positioned such that leg cutout edge 130a is disposed between front side edge 120a and back side edge 140a, and leg cutout edge 130b is disposed between front side edge 120b and 140b.

Referring still to FIG. 4, the absorbent article further comprises two inner leg gathers 2a and 2b, each of which extends longitudinally along the topsheet of the absorbent article from the back edge 110 to the front edge 100. The inner leg gathers 2a and 2b are approximately parallel to one another and are each positioned approximately parallel to the front and back side edges 120a, 120b, 140a and 140b in the longitudinal direction of the absorbent article. Further, each inner leg gather is located adjacent to and inward of the inner most portion of the leg cutout edges 130a and 130b.

Referring still to FIG. 4, the absorbent article further comprises two leg elastics 4a and 4b, each of which is positioned approximately adjacent and parallel to each of the inner leg gathers 2a and 2b. Each of the leg elastics is located outward of said inner leg gathers 2a and 2b. Two fasteners 6a and 6b are each located at or near the back side edge of the absorbent article. An absorbent core 8 is shown as being disposed between the topsheet 40 and a substantially impermeable backsheet 50. The absorbent core 8 is positioned longitudinally along the absorbent article. Waist foam 10 is optionally present in the absorbent article and positioned near and adjacent to the front edge 100 and/or back edge 110, disposed between the topsheet 40 and the backsheet 50.

As shown in FIG. 4, a hydrophilic zone 200 is shown as one contiguous area being located on the topsheet 40 inward of the front edge 100 and positioned centrally between the two inner leg gathers 2a and 2b, as well as the outside edges of the absorbent core, and corresponding to a female and a male insult point. Also shown adjacent to the hydrophilic zone 200 on the topsheet 40 is a hydrophobic zone 202. The hydrophobic zone 202 encompasses every portion of the topsheet not indicated as the hydrophilic zone 200. The hydrophilic zone 200 is shaped in a manner adapted to achieve maximum efficiency in a unisex diaper (e.g., a diaper suitable for both male and female babies). A person of skill in the art would readily be able to adapt the hydrophilic zone, based upon the guidance provided herein.

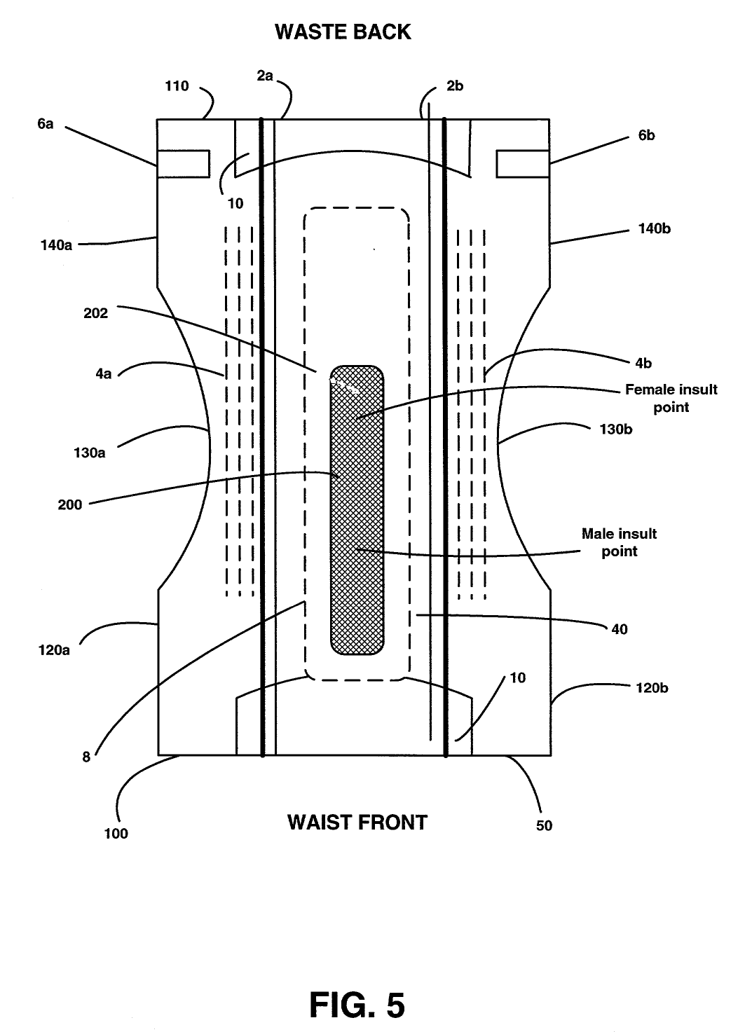

Referring to FIG. 5, an absorbent article (e.g., a diaper) is shown from a topsheet perspective. The absorbent article comprises a front edge 100, a back edge 110, two front side edges 120a and 120b, two back side edges 140a and 140b, and two leg cutout edges 130a and 130b, positioned such that leg cutout edge 130a is disposed between front side edge 120a and back side edge 140a, and leg cutout edge 130b is disposed between front side edge 120b and 140b.

Referring still to FIG. 5, the absorbent article further comprises two inner leg gathers 2a and 2b, each of which extends longitudinally along the topsheet of the absorbent article from the back edge 110 to the front edge 100. The inner leg gathers 2a and 2b are approximately parallel to one another and are each positioned approximately parallel to the front and back side edges 120a, 120b, 140a and 140b in the longitudinal direction of the absorbent article. Further, each inner leg gather is located adjacent to and inward of the inner most portion of the leg cutout edges 130a and 130b.

Referring still to FIG. 5, the absorbent article further comprises two leg elastics 4a and 4b, each of which is positioned approximately adjacent and parallel to each of the inner leg gathers 2a and 2b. Each of the leg elastics is located outward of said inner leg gathers 2a and 2b. Two fasteners 6a and 6b are each located at or near the back side edge of the absorbent article. An absorbent core 8 is shown as being disposed between the topsheet 40 and a substantially impermeable backsheet 50. The absorbent core 8 is positioned longitudinally along the absorbent article. Waist foam 10 is optionally present in the absorbent article and positioned near and adjacent to the front edge 100 and/or back edge 110, disposed between the topsheet 40 and the backsheet 50.

As shown in FIG. 5, a hydrophilic zone 200 is shown being located on the topsheet 40 inward of the front edge 100 and positioned centrally between the two inner leg gathers 2a and 2b, as well as the outside edges of the absorbent core, and corresponding to female and male insult points. Also shown adjacent to the hydrophilic zone 200 on the topsheet 40 is a hydrophobic zone 202. The hydrophobic zone 202 encompasses every portion of the topsheet not indicated as the hydrophilic zone 200.

In accordance with an implementation of the present invention, the hydrophilic zone comprises inherently hydrophobic material that has been treated with a composition that renders the material substantially hydrophilic. The composition may comprise any substance that is substantially effective in rendering substantially inherently hydrophobic material substantially hydrophilic and that would be safe for use on a material that will come into contact with human skin (e.g., would not cause irritation or damage). Preferably, the substance is a surfactant. Non-limiting exemplary surfactants include TRITON GR-5M, TRITON X-200 and TRITON X-100, available from Union Carbide Corp., Danbury, Conn., SILASTOL PST, available from Schill and Seilacher, Boblingen, Germany, SYNTHESIN FPC, available from Boehme Filatex, Inc., AHCOVEL, available from ICI Atkemix, Brantford, Ontario, and combinations thereof.

The substance may be a skin-wellness substance. A skin-wellness substance, as referred to herein, may be any substance having or perceived as having a benefit to skin when said substance comes into contact with the skin. As such, the present invention contemplates the use of substances effective for skin protection, skin care, skin wellness, skin improvement, substances generally perceived as having a skin wellness benefit or combinations thereof, for example, without limitation. Persons of ordinary skill in the art would readily be able to identify and select such substances for use in implementations of the present invention, based upon the disclosure herein.

Further, the skin-wellness substances of the present invention may include any of the following: a substance that that renders the inherently hydrophobic material substantially hydrophilic, a substance that when added to one or more other substances renders the inherently hydrophobic material substantially hydrophilic or a substance that can be added to one or more other substances that render the inherently hydrophobic material substantially hydrophilic without substantially compromising the effectiveness of the substance or substances, for example, without limitation.

Non-limiting exemplary skin-wellness substances include Aloe vera (aloe barbadensis), Bisabolol, Calendula Officinalis, Canadian Balsam (Abies balsamea), Cocoa Butter (Theobroma Cacao seed butter), Eucalyptus leaf oil (Eucalyptus globulus), German chamomile (Matricaria recutica), Gorgonian extract (Pseudotergoria elisabethae), Jojoba, Lanolin, Lavandin (Lavandula X intermedia), Lemon balm (Melissa officinalis), Rosemary (Rosmarinus officinalis), Spike lavender (Lavandula latifolia), Tea tree (Melaleuca altemifolia), True Lavender (Lavandula angustifolia), Vitamin E, Vitamin E-acetate, Witch Hazel (Hamamelis Virginiana) and any combinations thereof

Preferably, the skin-wellness substance is selected from the group consisting of Rosemary Oil (Rosmarinus officinalis), Cocoa Butter (Theobroma Cacao seed butter), Eucalyptus leaf oil (Eucalyptus globulus), True Lavender (Lavandula angustifolia), Tea tree oil (Melaleuca alternifolia), German chamomile (Matricaria recutica), Bisabolol, Aloe vera (aloe barbadensis) and combinations thereof. More preferably, the skin-wellness substance is selected from the group consisting of Tea tree oil (Melaleuca alternifolia), German chamomile (Matricaria recutica), Bisabolol, Aloe vera (aloe barbadensis) and combinations thereof. Even more preferably, the skin-wellness substance is Aloe vera (aloe barbadensis).

The substance for rendering the inherently hydrophobic material substantially hydrophilic, the skin-wellness substance or combinations thereof may be uniform within a treated hydrophilic zone or may be independently varied within a treated hydrophilic zone and/or may vary between separate contiguous areas of the a hydrophilic zone.

Where the hydrophilic zone includes multiple separate contiguous areas, in accordance with an implementation of the present invention, the substance for treating each area may comprise the same substance or combination of substances, or one or more of the areas may comprise a different substance or combination of substances.

The surfactant and/or skill wellness substance may partially or totally cover the treated bydrophilic zone. Whether the surfactant and/or skill wellness substance covers the entire treated hydrophilic zone or partially covers the treated hydrophilic zone may be determined by an optimization of cost-efficiency and effectiveness, as would be readily determined by a person of skill in the art.

The substances applied to the nonwoven may comprise any appropriate additive for absorbent articles. For example, stabilizers, excipients, colorants, fragrances and the like, are contemplated as additives by the present invention. Persons of ordinary skill in the art would be readily able to identify and select such additives for use in implementations of the present invention, based upon the disclosure herein.

The untreated hydrophobic zone may be of various sizes and shapes on the permeable topsheet that are effective in providing leak protection. The general shape and size is determined by the placement, shape and size of the treated hydrophilic zone and the overall size of the topsheet.

The treated hydrophilic zone is defined by an area that corresponds to a predetermined insult point or plurality of insult points. The treated hydrophilic zone forms a shaped pattern or plurality of shaped patterns on the selectively-permeable topsheet to substantially correspond to an insult point or plurality of predetermined insult points.

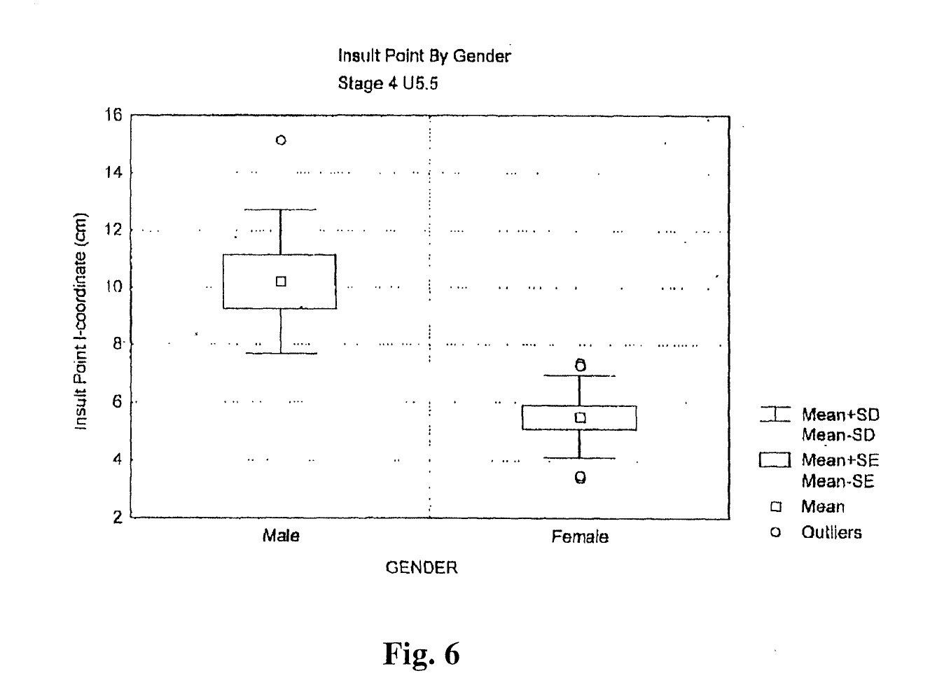

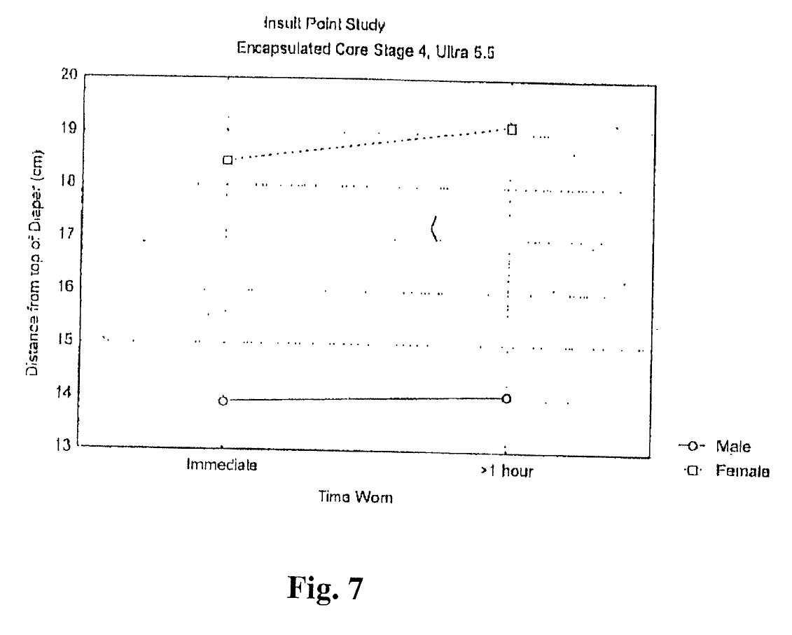

FIGS. 6 and 7 show graphs that illustrate the results from the study to determine the site of insult points for male and female babies. In particular, FIG. 6 shows the results of the insult points study for male and female babies as a function of gender and distance from the diaper fold in centimeters (cm). As shown in the figure, the male insult points are on average about 10.3 cm from the fold (where i,j is 0,0) of the diaper and the female insult points are on average about 5.6 cm from the fold of the diaper. Moreover, according to the study, male insult points are more variable than female insult points. This is shown in the figure by the standard deviation for males which equals about 2.5 cm as opposed to the standard deviation for females, which equals about 1.5 cm. A diaper having a substantially similar absorbency at the male and female insult points is obtained in accordance with one implementation of the invention. In particular, a core may be formed having a DI index at the male and female insult points which is substantially similar, as described above. In this manner, the present invention provides a superior unisex diaper (e.g., a diaper which is optimal for males and females). As used herein male insult point refers to average male insult point and female insult point refers to average female insult point. Persons of ordinary skill in the art appreciate that the insult point will vary as a function of age of the child. Accordingly, the present invention contemplates adjustments to tailor the absorbent article to children of different ages, which is well within the skill of the art.

FIG. 7 shows the results of the insult points study for males and females with regard to variations and insult points over time. The graph in FIG. 7 shows the changes in insult point as a function of time and distance from the top of the diaper (cm). As the graph illustrates, over time the insult point appears to move up. This is likely due to sagging of the diaper.

In accordance with an implementation of the invention, changes in position of the insult point caused by sagging can be accounted for by tailoring a Distribution Index Profile of an absorbent article in accordance with the known change in position of the insult points that occur when the diaper begins sagging. In particular, the DI may be substantially similar in the area around and between the original insult point and the post-sagging insult point. Thus, absorbency is provided in the areas of greatest need during the course of use of the diaper by the wearer. In this manner, an absorbent article having superior longevity may be provided by the present invention. This would be particularly useful, for example, for an extended-use diaper, a nighttime diaper (e.g., a diaper intended primarily for use during the nighttime) and/or a travel diaper (e.g., a diaper intended primary for use during travel or a long road trip, or during any somewhat long period of time during which changing the diaper would be generally inconvenient), without limitation.

The hydrophilic zone may be positioned anywhere on the topsheet provided is substantially corresponds to the intended insult point or plurality of predetermined insult points. Preferably, the topsheet is positioned centrally in the transverse direction on the selectively-permeable topsheet.

The treated hydrophilic zone may be of any suitable shape or size as would be readily determined by a person of skill in the art based upon the guidance provided herein. Preferably, the hydrophilic zone is an approximately oval shaped area of about 118 mm to about 144 mm in width at the widest portion and about 260 mm to about 407 mm in length at the longest portion, more preferably the hydrophilic zone is an approximately oval shaped area of about 113 mm to about 139 mm in width at the widest portion and about 250 mm to about 397 mm in length at the longest portion, and even more preferably the hydrophilic zone is an approximately oval shaped area of about 108 mm to about 134 mm in width at the widest portion and about 240 mm to about 387 mm in length at the longest portion.

The hydrophilic zone may be placed in any suitable location on the topsheet as would be readily determined by a person of skill in the art based upon the guidance provided herein. Preferably, the forward most point of the hydrophilic zone is about 0 mm to about 45 mm from the front edge of the absorbent core and the rear most point of the hydrophilic zone is about 0 mm to about 45 mm from the back edge of the absorbent core, more preferably the forward most point of the hydrophilic zone is about 0 mm to about 60 mm from the front edge of the absorbent core and the rear most point of the hydrophilic zone is about 0 mm to about 60 mm from the back edge of the absorbent core, and even more preferably the forward most point of the hydrophilic zone is about 0 mm to about 75 mm from the front edge of the absorbent core and the rear most point of the hydrophilic zone is about 0 mm to about 75 mm from the back edge of the absorbent core.

According to an implementation of the present invention, the treated hydrophilic zone corresponds to a female insult point or range of female insult points on said selectively-permeable topsheet. In this implementation, preferably the treated hydrophilic zone is positioned centrally in the transverse direction and the forward most point on the hydrophilic zone is about 35 mm to about 55 mm from the front edge of the absorbent core and the rear most point of the hydrophilic zone is about 0 mm to about 55 mm from the back edge of the absorbent core, more preferably the treated hydrophilic zone is positioned centrally in the transverse direction and the forward most point on the hydrophilic zone is about 35 mm to about 75 mm from the front edge of the absorbent core and the rear most point of the hydrophilic zone is about 0 mm to about 75 mm from the back edge of the absorbent core, and even more preferably the treated hydrophilic zone is positioned centrally in the transverse direction and the forward most point on the hydrophilic zone is about 35 mm to about 90 mm from the front edge of the absorbent core and the rear most point of the hydrophilic zone is about 0 mm to about 90 mm from the back edge of the absorbent core.

According to an implementation of the present invention , the treated hydrophilic zone corresponds to a male insult point or range of male insult points on said selectively-permeable topsheet. According to this implementation, preferably the treated hydrophilic zone is positioned centrally in the transverse direction and the forward most point on the hydrophilic zone is about 0 mm to about 35 mm from the front edge of the absorbent core and the rear most point of the hydrophilic zone is about 0 mm to about 55 mm from the back edge of the absorbent core, more preferably the treated hydrophilic zone is positioned centrally in the transverse direction and the forward most point on the hydrophilic zone is about 0 mm to about 45 mm from the front edge of the absorbent core and the rear most point of the hydrophilic zone is about 0 mm to about 75 mm from the back edge of the absorbent core, and even more preferably the treated hydrophilic zone is positioned centrally in the transverse direction and the forward most point on the hydrophilic zone is about 0 mm to about 65 mm from the front edge of the absorbent core and the rear most point of the hydrophilic zone is about 0 mm to about 90 mm from the back edge of the absorbent core.

As noted herein, the shape and size of the hydrophilic zone is general determined in a manner as to correspond with the predetermined insult point or plurality of insult points in order to attain superior leak protection. The maximum width (the distance from one side edge to the other, in the transverse direction) of the treated hydrophilic zone (or each contiguous area of the hydrophilic zone) is preferably about 118 mm to about 144 mm, more preferably about 113 mm to about 139 mm, or even more preferably about 108 mm to about 134 mm. The maximum length (the distance from the front edge to the back edge, in the longitudinal direction) of the treated hydrophilic zone (or each contiguous area of the hydrophilic zone) is preferably about 260 mm to about 407 mm, more preferably about 250 mm to about 397 mm, or even more preferably about 240 mm to about 387 mm.

The substances used to treat the nonwoven may be of any substantially effective density. The substances may be a substantially uniform density or a non-uniform density. Moreover, the density of the substances may vary from one contiguous area to another. Preferably, the density of the hydrophobic composition is selected to optimize between costefficiency and effectiveness of the absorbent article, and/or method for preparing or using same. Different materials will require different densities as would be known to a person of skill in the art. Preferably, the density of said hydrophilic composition is about 0.05 mg/m² to about 10 mg/M²; more preferably about 0.05 mg/m² to about 100 mg/m²; and even more preferably about 0.05 mg/m² to about 1,000 mg/m².

The absorbent core may be of any desirable shape. Non-limiting exemplary shapes of absorbent cores in accordance with the present invention include a rectangular shape, a substantially rectangular shape, a T shape, an hour glass shape, or combinations thereof. Preferably, the absorbent core is a rectangular shape. Preferably, the absorbent core is approximately rectangular in shape.

Due to the wide variety of materials which may be incorporated into the absorbent articles of the present invention, the present invention is not intended to be limited to any specific materials. The topsheet, backsheet, absorbent core and other components of the absorbent articles, in accordance with various implementations of the present invention, may comprise various materials. Persons of ordinary skill in the art would be readily able to select appropriate materials for use in the various components of the present invention based upon the guidance provided herein.

In accordance with various implementations of the present invention, the absorbent core may contain one or more fibers, one or more polymers or combinations thereof. Nonlimiting exemplary fibers which may be used in the articles of the present invention include, without limitation, cellulose fibers, cellulose acetate fibers, rayon fibers, Courtauld's LYOCEL fibers, polyacrylonitrile fibers, surface modified (hydrophilic) polyester fibers, surface modified polyolophin/polyester bicomponent fibers, surface modified polyester/polyester bicomponent fibers, cotton fibers or blends thereof. Preferably cellulose acetate, rayon, Courtauld's LYOCEL, polyacrylonitrile, cotton fibers and cotton linters or combinations thereof are used in the process of the present invention. More preferably, cellulose fibers are used as the fiber material in the present invention.

Other materials may be added to the fiber or pulp material which is optionally processed in a fiberizing apparatus, such as a hammermill. The additives may be added at any point in the process. Preferably, the additives are sprayed or injected into the airborne fibers prior to the depositing of the fibers on the forming surface 2. Non-limiting exemplary additives which may be incorporated into the process of the present invention include a polymer such as a super absorbent polymer (SAP), hydrophilic polymers, potato starch, corn starch, wheat starch or rice starch, or combinations thereof. Various different combinations of materials may be used as are known to persons of ordinary skill in the art and which are described in U.S. Pat. No. 6,068,620 which is herein incorporated by reference. Preferably, the mixtures incorporated in the invention are substantially homogenous mixtures or uniformly distributed mixtures. Absorbent articles in accordance with an implementation of the present invention are prepared using conventional methods and materials well known to persons of ordinary skill in the art, using the guidelines provided herein. In one implementation of the present invention an absorbent article is prepared by a process comprising: identifying one or more hydrophobic zones on an absorbent article; applying a hydrophobic composition to a permeable topsheet at the one or more hydrophobic zones on said permeable topsheet; and forming an absorbent article comprising said permeable topsheet, a substantially impermeable backsheet and an absorbent core disposed between said permeable topsheet and said substantially impermeable backsheet.

The present invention further provides methods for preparing absorbent articles having selectively-permeable topsheets with a treated hydrophilic zone to provide superior leakage protection. An implementation of the present invention provides a method for preparing an absorbent article comprising: disposing a composition on a nonwoven at one or more predetermined area corresponding to an insult point or plurality of insult points to form a selectively-permeable topsheet having a treated hydrophilic zone or plurality of treated hydrophilic zones corresponding to the one or more predetermined areas, said nonwoven being inherently hydrophobic and comprising fibers or film.

The composition used in the method of the present invention to treat the nonwoven to create the hydrophilic zone comprises a substance for rendering an inherently hydrophobic material substantially hydrophilic, such as a surfactant, for example. The composition may also include a skin-wellness substance, such as any substance effective or perceived as being effective in providing skin protection, skin care, skin improvement, or any combination thereof, for example.

Preferably, the skin-wellness substance is selected from the group consisting of Aloe (aloe barbadensis), Bisabolol, Calendula Officinalis, Canadian Balsam (Abies balsamea), Cocoa Butter (Theobroma Cacao seed butter), Eucalyptus leaf oil (Eucalyptus globulus), German chamomile (Matricaria recutica), Gorgonian extract (Pseudotergoria elisabethae), Jojoba, Lanolin, Lavandin (Lavandula X intermedia), Lemon balm (Melissa officinalis), Rosemary (Rosmarinus officinalis), Spike lavender (Lavandula latifolia), Tea tree (Melaleuca alternifolia), True Lavender (Lavandula angustifolia), Vitamin E, Vitamin E-acetate, Witch Hazel (Hamamelis Virginiana) and combinations thereof.

According to an implementation of the present invention, the area (or areas) to be treated may be treated in line during the fabrication of the absorbent article. The treated area (or areas) which form the hydrophilic zone is defined by an area that corresponds to a predetermined insult point or plurality of insult points. The area (or areas) may form a variety of shaped patterns or plurality of shaped patterns on the selectively-permeable topsheet to substantially correspond to an insult point or plurality of predetermined insult points.

Non-limiting exemplary methods of applying the substance or substances used to treat the nonwoven include spraying, aerosolizing, coating, painting, pressing, layering, disbursing and/or combinations thereof. Preferably, the method of applying the substance or substances used to treat the nonwoven would involve aerosolizing. A person of ordinary skill in the art would readily be able to practice the methods of the present invention using said conventional methods and techniques, including selecting the most appropriate methods and techniques, based upon the guidance provided in the disclosure herein, without undue experimentation.

The present invention also provides compositions for use in rendering inherently hydrophobic material hydrophilic while providing skin-wellness benefits, as described herein. According to an implementation, the present invention provides a composition in an absorbent article for providing leak protection and improved skin wellness comprising: an amount of surfactant sufficient for rendering substantially hydrophilic an inherently hydrophobic material comprising nonwoven fibers or films; and a skin wellness substance selected from the group consisting of Aloe vera (aloe barbadensis), Bisabolol, Calendula Officinalis, Canadian Balsam (Abies balsamea), Cocoa Butter (Theobroma Cacao seed butter), Eucalyptus leaf oil (Eucalyptus globulus), German chamomile (Matricaria recutica), Gorgonian extract (Pseudotergoria elisabethae), Jojoba, Lanolin, Lavandin (Lavandula X intemedia), Lemon balm (Melissa officinalis), Rosemary (Rosmarinus officinalis), Spike lavender (Lavandula latifolia), Tea tree (Melaleuca alternifolia), True Lavender (Lavandula angustifolia), Vitamin E, Vitamin E-acetate, Witch Hazel (Hamamelis Virginiana) and combinations thereof. Preferably, the surfactant is selected from the group consisting of TRITON GR-5M, TRITON X-200 and TRITON X-100, available from Union Carbide Corp., Danbury, Conn., and SILASTOL PST, available from Schill and Seilacher, Boblingen, Germany, and combinations thereof.

The present invention has been described in connection with the preferred embodiments. These embodiments, however, are merely for example and the invention is not restricted thereto. Any examples described herein are illustrative of preferred embodiments of the inventive subject matter and are not to be construed as limiting the inventive subject matter thereto. It will be understood by those skilled in the art that other variations and modifications can easily be made within the scope of the invention as defined by the appended claims.

What is claimed is:

1. An absorbent article comprising:
a selectively-permeable topsheet;
a substantially impermeable backsheet; and
an absorbent core disposed between said selectively-permeable topsheet and said substantially impermeable backsheet;
wherein the selectively-permeable topsheet comprises at least one treated hydrophilic zone and at least one non-treated hydrophobic zone.

2. The absorbent article of claim 1, wherein the treated hydrophilic zone comprises a surfactant.

3. The absorbent article of claim 1, wherein the treated hydrophilic zone comprises a skin-wellness substance.

4. The absorbent article of claim 3, wherein the skin-wellness substance is any substance effective or perceived as being effective in providing skin protection, skin care, skin improvement, or any combination thereof.

5. The absorbent article of claim 3, wherein the skin-wellness substance is selected from the group consisting of Aloe (aloe barbadensis), Bisabolol, Calendula Officinalis, Canadian Balsam (Abies balsamea), Cocoa Butter (Theobroma Cacao seed butter), Eucalyptus leaf oil (Eucalyptus globulus), German chamomile (Matricaria recutica), Gorgonian extract (Pseudotergoria elisabethae), Jojoba, Lanolin, Lavandin (Lavandula X intermedia), Lemon balm (Melissa officinalis), Rosemary (Rosmarinus officinalis), Spike lavender (Lavandula latifolia), Tea tree (Melaleuca alternifolia), True Lavender (Lavandula angustifolia), Vitamin E, Vitamin E-acetate, Witch Hazel (Hamamelis Virginiana) and combinations thereof.

6. The absorbent article of claim 3, wherein the skin-wellness substance is Aloe.

7. The absorbent article of claim 3, wherein the skin-wellness substance is Vitamin E.

8. The absorbent article of claim 3, wherein the skin-wellness substance is Cocoa Butter.

9. The absorbent article of claim 1, wherein the treated hydrophilic zone comprises a surfactant and a skin wellness substance.

10. The absorbent article of claim 1, wherein the treated hydrophilic zone is defined by an area that corresponds to a predetermined insult point or plurality of insult points.

11. The absorbent article of claim 1, wherein the treated hydrophilic zone forms a shaped pattern or plurality of shaped patterns on the selectively-permeable topsheet to substantially correspond to an insult point or plurality of predetermined insult points.

12. The absorbent article of claim 1, wherein the hydrophilic zone is positioned centrally in the transverse direction on the selectively-permeable topsheet.

13. The absorbent article of claim 1, wherein the treated hydrophilic zone is an approximately oval shaped area of about 118 mm to about 144 mm in width at the widest portion and about 260 mm to about 407 mm in length at the longest portion.

14. The absorbent article of claim 1, wherein the forward most point of the hydrophilic zone is about 0 mm to about 20 mm from the front edge of the absorbent core and the rear most point of the hydrophilic zone is about 0 mm to about 20 mm from the back edge of the absorbent core.

15. The absorbent article of claim 1, wherein the treated hydrophilic zone corresponds to a male insult point or range of male insult points on said selectively-permeable topsheet.

16. The absorbent article of claim 15, wherein the treated hydrophilic zone is positioned centrally in the transverse direction and the forward most point on the hydrophilic zone is about 0 mm to about 35 mm from the front edge of the absorbent core.

17. The absorbent article of claim 1, wherein the treated hydrophilic zone corresponds to a female insult point or range of female insult points on said selectively-permeable topsheet.

18. The absorbent article of claim 17, wherein the treated hydrophilic zone is positioned centrally in the transverse direction and the forward most point on the hydrophilic zone is about 35 mm to about 55 mm from the front edge of the absorbent core and the rear most point of the hydrophilic zone.

19. An absorbent a rticle comprising:
a selectively-permeable topsheet;
a substantially impermeable backsheet; and
an absorbent core disposed between said selectively-permeable topsheet and said substantially impermeable backsheet;
wherein the selectively-permeable topsheet comprises at least one treated hydrophilic zone and at least one non-treated hydrophobic zone, said treated hydrophilic zone comprising a surfactant and a skin wellness ingredient; and
wherein the treated hydrophilic zone is defined by an area that corresponds to a predetermined insult point or plurality of insult points.

20. The absorbent article of claim 19, wherein the skin-wellness substance is selected from the group consisting of Aloe (aloe barbadensis), Bisabolol, Calendula Officinalis, Canadian Balsam (Abies balsamea), Cocoa Butter (Theobroma Cacao seed butter), Eucalyptus leaf oil (Eucalyptus globulus), German chamomile (Matricaria recutica), Gorgonian extract (Pseudotergoria elisabethae), Jojoba, Lanolin, Lavandin (Lavandula X intermedia), Lemon balm (Melissa officinalis), Rosemary (Rosmarinus officinalis), Spike lavender (Lavandula latifolia), Tea tree (Melaleuca alternifolia), True Lavender (Lavandula angustifolia), Vitamin E, Vitamin E-acetate, Witch Hazel (Hamamelis Virginiana) and combinations thereof.

21. The absorbent article of claim 19, wherein the treated hydrophilic zone forms a shaped pattern or plurality of shaped patterns on the selectively-permeable topsheet.

22. The absorbent article of claim 19, wherein the hydrophilic zone is positioned centrally in the transverse direction on the selectively-permeable topsheet.

23. The absorbent article of claim 19, wherein the treated hydrophilic zone is an approximately oval shaped area of about 118 mm to about 144 mm in width at the widest portion and about 260 mm to about 407 mm in length at the longest portion.

24. The absorbent article of claim 19, wherein the forward most point of the hydrophilic zone is about 0 mm to about 20 mm from the front edge of the absorbent core and the rear most point of the hydrophilic zone is about 0 mm to about 20 mm from the back edge of the absorbent core.

25. The absorbent article of claim 19, wherein the treated hydrophilic zone corresponds to a male insult point or range of male insult points on said selectively-permeable topsheet.

26. The absorbent article of claim 25, wherein the treated hydrophilic zone is positioned centrally in the transverse direction and the forward most point on the hydrophilic zone is about 0 mm to about 35 mm from the front edge of the absorbent core.

27. The absorbent article of claim 19, wherein the treated hydrophilic zone corresponds to a female insult point or range of female insult points on said selectively-permeable topsheet.

28. The absorbent article of claim 27, wherein the treated hydrophilic zone is positioned centrally in the transverse direction and the forward most point on the hydrophilic zone is about 35 mm to about 55 mm from the front edge of the absorbent core.

29. An absorbent garment comprising:
a front and a rear waist portion cooperating to form a waist opening;
a crotch region formed between said front waist portion and said rear waist portion;
a pair of leg openings on opposed sides of the crotch region; and
a selectively permeable topsheet, a substantially impermeable backsheet and an absorbent core positioned between said selectively-permeable topsheet and said backsheet;
wherein the selectively-permeable topsheet comprises at least one treated hydrophilic zone corresponding to a portion or plurality of portions at the crotch region and at least one non-treated hydrophobic zone.

30. The absorbent garment of claim 29, further comprising a first leg gather and a second leg gather.

31. The absorbent garment of claim 29, wherein the hydrophilic zone is located on the selectively-permeable topsheet in an area between the first leg gather and the second leg gather.

32. The absorbent garment of claim 31, wherein the hydrophilic zone is centrally located in the transverse direction between the first leg gather and the second leg gather.

33. A method for preparing an absorbent article comprising:
disposing a composition on a nonwoven at one or more predetermined area corresponding to an insult point or plurality of insult points to form a selectively-permeable topsheet having a treated hydrophilic zone or plurality of treated hydrophilic zones corresponding to the one or more predetermined areas, said nonwoven being inherently hydrophobic and comprising fibers or film.

34. The method of claim 33, wherein the composition comprises a surfactant.

35. The method of claim 33, wherein the composition comprises a skin-wellness substance.

36. The method of claim 35, wherein the skin-wellness substance is any substance effective or perceived as being effective in providing skin protection, skin care, skin improvement, or any combination thereof.

37. The method of claim 35, wherein the skin-wellness substance is selected from the group consisting of Aloe (aloe barbadensis), Bisabolol, Calendula Officinalis, Canadian Balsam (Abies balsamea), Cocoa Butter (Theobroma Cacao seed butter), Eucalyptus leaf oil (Eucalyptus globulus), German chamomile (Matricaria recutica), Gorgonian extract (Pseudotergoria elisabethae), Jojoba, Lanolin, Lavandin (Lavandula X intermedia), Lemon balm (Melissa officinalis), Rosemary (Rosmarinus officinalis), Spike lavender (Lavandula latifolia), Tea tree (Melaleuca alternifolia), True Lavender (Lavandula angustifolia), Vitamin E, Vitamin E-acetate, Witch Hazel (Hamamelis Virginiana) and combinations thereof.

38. The method of claim 35, wherein the skin-wellness substance is Aloe.

39. The method of claim 35, wherein the skin-wellness substance is Vitamin E.

40. The method of claim 35, wherein the skin-wellness substance is Cocoa Butter.

41. The method of claim 33, wherein the treated hydrophilic zone comprises a surfactant and a skin wellness substance.

42. The method of claim 33, wherein the treated hydrophilic zone is defined by an area that corresponds to a predetermined insult point or plurality of insult points.

43. The method of claim 33, wherein the treated hydrophilic zone forms a shaped pattern or plurality of shaped patterns on the selectively-permeable topsheet to substantially correspond to an insult point or plurality of predetermined insult points.

44. The method of claim 33, wherein the hydrophilic zone is positioned centrally in the transverse direction on the selectively-permeable topsheet.

45. The method of claim 33, wherein the treated hydrophilic zone is an approximately oval shaped area of about 118 mm to about 144 mm in width at the widest portion and about 260 mm to about 407 mm in length at the longest portion.

46. The method of claim 33, wherein the forward most point of the hydrophilic zone is about 0 mm to about 20 mm from the front edge of the absorbent core and the rear most point of the hydrophilic zone is about 0 mm to about 20 mm from the back edge of the absorbent core.

47. The method of claim 33, wherein the treated hydrophilic zone corresponds to a male insult point or range of male insult points on said selectively-permeable topsheet.

48. The method of claim 47, wherein the treated hydrophilic zone is positioned centrally in the transverse direction and the forward most point on the hydrophilic zone is about 0 mm to about 35 mm from the front edge of the absorbent core.

49. The method of claim 33, wherein the treated hydrophilic zone corresponds to a female insult point or range of female insult points on said selectively-permeable topsheet.

50. The method of claim 49, wherein the treated hydrophilic zone is positioned centrally in the transverse direction and the forward most point on the hydrophilic zone is about 35 mm to about 55 mm from the front edge of the absorbent core.

51. A composition in an absorbent article for providing leak protection and improved skin wellness comprising:
an amount of surfactant sufficient for rendering substantially hydrophilic an inherently hydrophobic material comprising nonwoven fibers or films; and
a skin wellness substance selected from the group consisting of Aloe (aloe barbadensis), Bisabolol, Calendula Officinalis, Canadian Balsam (Abies balsamea), Cocoa Butter (Theobroma Cacao seed butter), Eucalyptus leaf oil (Eucalyptus globulus), German chamomile (Matricaria recutica), Gorgonian extract (Pseudotergoria elisabethae), Jojoba, Lanolin, Lavandin (Lavandula X intermedia), Lemon balm (Melissa officinalis), Rosemary (Rosmarinus officinalis), Spike lavender (Lavandula latifolia), Tea tree (Melaleuca alternifolia), True Lavender (Lavandula angustifolia), Vitamin E, Vitamin E-acetate, Witch Hazel (Hamamelis Virginiana) and combinations thereof.

52. The composition of claim 51, wherein the surfactant is selected from the group consisting of TRITON GR-5M, TRITON X-200 and TRITON X-100, available from Union Carbide Corp., Danbury, Conn., and SILASTOL PST, available from Schill and Seilacher, Boblingen, Germany, and combinations thereof.
